(19) United States
(12) Patent Application Publication
(10) Pub. No.: US 2023/0279445 A1
(43) Pub. Date: Sep. 7, 2023

(54) METHANE PURIFICATION METHOD AND METHANE PURIFICATION APPARATUS

(71) Applicant: HONDA MOTOR CO., LTD., Tokyo (JP)

(72) Inventors: Minoru Goto, Wako-shi (JP); Nozomi Shiobara, Wako-shi (JP); Shohei Kinoshita, Wako-shi (JP); Mizuho Doi, Wako-shi (JP); Kenji Machida, Wako-shi (JP); Satoshi Shiozaki, Wako-shi (JP)

(21) Appl. No.: 18/114,296

(22) Filed: Feb. 27, 2023

(30) Foreign Application Priority Data
Mar. 1, 2022 (JP) 2022-030603

(51) Int. Cl. C12P 5/02; C12M 1/107; C12M 1/00; C12N 1/12

(52) U.S. Cl. CPC C12P 5/023; C12M 21/04; C12M 21/02; C12M 23/58; C12M 29/06; C12M 47/18; C12N 1/12

(57) ABSTRACT

A mixed gas generated in a methane fermentation tank is supplied to culture tanks. In the culture tanks, water as a culture solution is held and algae are cultured. Carbon dioxide contained in the mixed gas preferentially dissolves in water. That is, carbon dioxide is removed from the mixed gas. As a result, a concentration of methane, which is one component of the mixed gas, increases. The mixed gas is re-supplied to another culture tank or the same culture tank.

### CROSS-REFERENCE TO RELATED APPLICATIONS

This application is based upon and claims the benefit of priority from Japanese Patent Application No. 2022-030603 filed on Mar. 1, 2022, the contents of which are incorporated herein by reference.

### BACKGROUND OF THE INVENTION

### Field of the Invention

The present invention relates to a methane purification method in which methane is purified from a mixed gas obtained by methane fermentation. The present invention also relates to a methane purification apparatus for purifying methane.

### Description of the Related Art

As described in JP S58-081787 A, methane is obtained through methane fermentation of organic residue. However, the resulting methane contains carbon dioxide as a by-product. Therefore, in order to obtain methane of high purity, it is necessary to separate carbon dioxide from methane.

JP 2010-088368 A proposes that a gas produced by methane fermentation be brought into contact with water. The solubility of carbon dioxide in water is greater than that of methane in water. Therefore, carbon dioxide is absorbed by water while methane is discharged without being absorbed by water. Thus, it is possible to separate carbon dioxide from methane by bringing the gas into contact with water.

Up to now, efforts to mitigate climate change or reduce its impact have been continuing. To accomplish this, research and development on reduction of carbon dioxide is being carried out. In view of the above, it is required to consume carbon dioxide produced in methane fermentation. In the technique described in JP 2010-088368 A, water in which carbon dioxide has been dissolved after the separation is supplied to an algae culture apparatus. Carbon dioxide is consumed through the photosynthesis of algae.

### SUMMARY OF THE INVENTION

The methane gas production equipment described in JP 2010-088368 A requires an absorption tower for bringing gas into contact with water. Therefore, large equipment is required. Capital investment also rises sharply. Thus, on the way to climate change mitigation or impact reduction, there are problems that equipment becomes large and capital investment is not easily reduced.

An object of the present invention is to solve the aforementioned problems.

According to one embodiment of the present invention, provided is a methane purification method of purifying methane from a mixed gas containing carbon dioxide and methane by removing the carbon dioxide from the mixed gas, including a methane fermentation step of performing methane fermentation for organic residue, a supply step of supplying a mixed gas containing methane and carbon dioxide discharged in the methane fermentation step to a culture tank containing water as a culture solution for culturing algae, a culture step of culturing the algae by fixing the carbon dioxide contained in the mixed gas to the algae, and a re-supply step of supplying to the culture tank or another culture tank the mixed gas that has passed through the culture solution to reduce the carbon dioxide contained in the mixed gas, wherein the carbon dioxide contained in the mixed gas is consumed by performing the re-supply step at least once.

According to another embodiment of the present invention, provided is a methane purification apparatus that purifies methane from a mixed gas containing carbon dioxide and methane by removing carbon dioxide from the mixed gas, the apparatus including a methane fermentation tank that performs methane fermentation for organic residue, a culture tank that holds water as a culture solution for culturing algae, a first supply line that supplies the culture tank with a mixed gas containing carbon dioxide and methane discharged from the methane fermentation tank, and a second supply line that supplies another culture tank different from the culture tank with the mixed gas that has passed through the culture solution.

In the present invention, the mixed gas once supplied to the algae and reduced in carbon dioxide is re-supplied to the algae. By this resupply, carbon dioxide in the mixed gas further reduces. Therefore, the purity of methane increases. That is, methane of high purity can be obtained. Further, carbon dioxide is fixed in algae. As a result, the present invention reduces carbon dioxide and thus contributes to climate change mitigation or impact reduction.

In addition, an absorption tower for absorbing the mixed gas into water is unnecessary. Therefore, it is possible to reduce the size of the methane purification apparatus. Further, it is easy to reduce capital investment.

The above and other objects, features, and advantages of the present invention will become more apparent from the following description when taken in conjunction with the accompanying drawings, in which a preferred embodiment of the present invention is shown by way of illustrative example.

### BRIEF DESCRIPTION OF THE DRAWINGS

FIG. 1 is a schematic system diagram of a methane purification apparatus according to a first embodiment of the present invention.

FIG. 2 is a schematic diagram of the configuration of a flash distiller constituting the methane purification apparatus.

FIG. 3 is a schematic flow of a methane purification method according to an embodiment of the present invention.

FIG. 4 is a schematic system diagram of an essential part of a methane purification apparatus according to a second embodiment of the present invention.

### DETAILED DESCRIPTION OF THE INVENTION

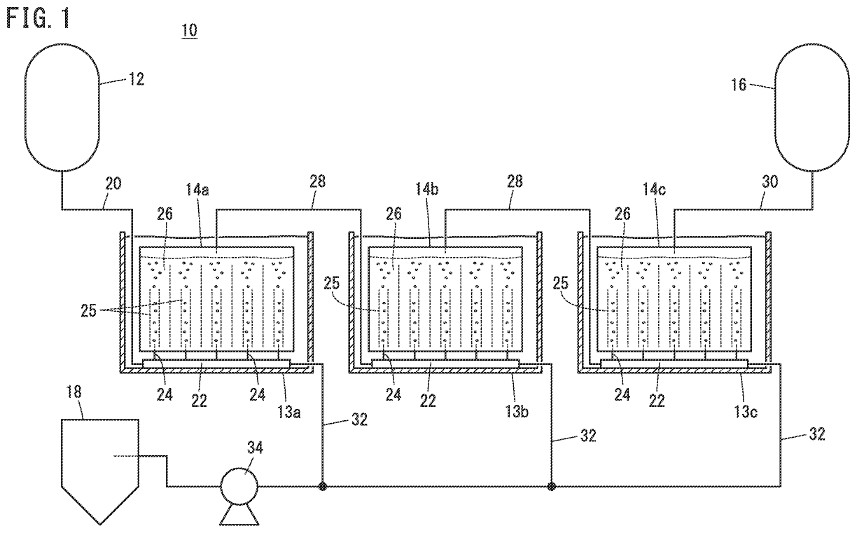

FIG. 1 is a schematic system diagram of a methane purification apparatus 10 according to a first embodiment. A methane purification apparatus 10 includes a methane fermentation tank 12, a plurality of culture tanks, a collection tank 16, and a flash distiller 18. In the illustrated example, the culture tanks are a first culture tank 14a, a second culture tank 14b, and a third culture tank 14c. The first culture tank 14a, the second culture tank 14b and the third culture tank 14c are housed in a first water tank 13a, a second water tank 13b and a third water tank 13c and are immersed in water.

In the methane fermentation tank 12, methane is produced from organic residue by methane producing bacteria. As the organic residue, cultured algae are used. Alternatively, sewage sludge or the like can be used as the organic residue. The methane fermentation tank 12 is, for example, a known fermentation tank such as the methane fermentation treatment apparatus described in JP 2015-024388 A.

The culture tank is a tank for culturing algae. In the first embodiment, for ease of understanding, a case where the number of culture tanks is three is shown as an example. Further, in order to easily distinguish the culture tanks from each other, each of the three culture tanks is referred to as the first culture tank 14a, the second culture tank 14b, and the third culture tank 14c.

A first supply line 20 is provided from the methane fermentation tank 12 to the first water tank 13a. The first supply line 20 is connected to a main pipe 22 in the first water tank 13a. The main pipe 22 extends horizontally at the bottom of the first water tank 13a. A plurality of sub-pipes 24 branch from the main pipe 22. The sub-pipes 24 extend in a substantially vertical direction.

A plurality of guide pipes 25 are provided inside the first culture tank 14a. The guide pipes 25 are disposed above the sub-pipes 24 and receive the gas (mixed gas) discharged from the sub-pipes 24. Water 26 is stored in the first culture tank 14a and the entire guide pipe 25 is immersed in the water 26. The water 26 is a culture solution (culture medium) for culturing algae.

A second supply line 28 is provided on a ceiling wall of the first culture tank 14a. The second supply line 28 extends horizontally and then bends vertically downward outside the first culture tank 14a.

The second culture tank 14b is configured similarly to the first culture tank 14a. Accordingly, the same components are denoted by the same reference numerals, and detailed description thereof will be omitted. The second supply line 28 of the first culture tank 14a is connected to the main pipe 22 inside the second culture tank 14b.

The third culture tank 14c is also configured similarly to the first culture tank 14a. The second supply line 28 of the second culture tank 14b is connected to the main pipe 22 inside the third culture tank 14c. A collection line 30 is provided at a ceiling wall of the third culture tank 14c. The collection line 30 is connected to the collection tank 16.

As understood from the above, the first culture tank 14a is located most upstream in the gas flow direction among the three culture tanks. The third culture tank 14c is located most downstream in the gas flow direction among the three culture tanks. The second culture tank 14b is located at a middle position between the first culture tank 14a and the third culture tank 14c.

The flash distiller 18 is a tower that performs flash distillation for the mixture of the cultured algae and the culture solution (water 26). Methane is separated from the mixture through flash distillation. The flash distiller 18 is in a state of being disconnected from the gas flow line.

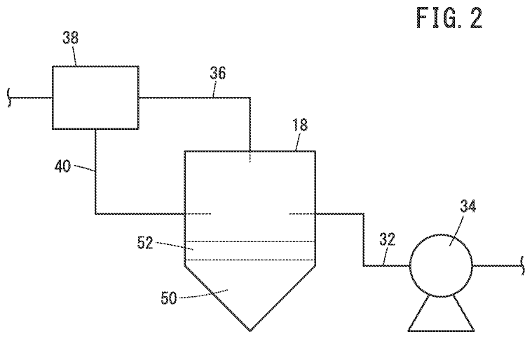

As shown in FIGS. 1 and 2, solution feeding lines 32 are provided between the flash distiller 18 and the first to third culture tanks 14a to 14c, respectively. The solution feeding line 32 is provided with a pump 34. Accordingly, the respective culture solution in the first to third culture tanks 14a to 14c are fed into the flash distiller 18 via the solution feed line 32 based on the driving of the pump 34.

As shown in FIG. 2, the flash distiller 18 is provided with an exhaust line 36. The exhaust line 36 is provided with a condenser 38. A return line 40 for returning the condensed culture solution to the flash distiller 18 is connected to the condenser 38.

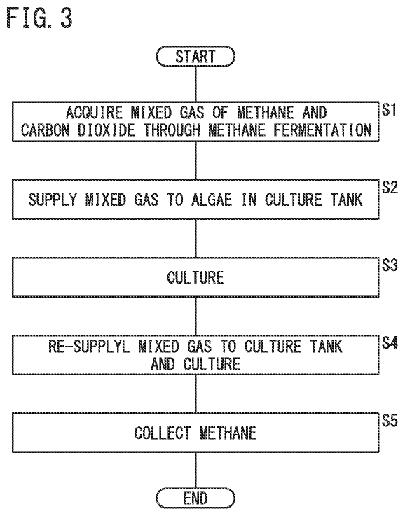

Next, a methane purification method according to the first embodiment will be described with reference to a schematic flow shown in FIG. 3. The purification method includes a methane fermentation step S1, a supply step S2, a culture step S3, a re-supply step S4, and a collection step S5.

In the methane fermentation step S1, methane fermentation is performed in the methane fermentation tank 12. Specifically, for example, with algae that have been cultured being used as organic residue, methane is produced by methane producing bacteria. Here, carbon dioxide is produced as a by-product during methane fermentation. That is, the gas actually produced is a mixed gas containing methane and carbon dioxide. Digestate produced as a by-product of methane fermentation contains inorganic substances such as phosphorus, nitrogen, and potassium.

In the first embodiment, carbon dioxide is removed from the mixed gas in order to obtain methane of high purity. Therefore, the mixed gas is sequentially passed through the first culture tank 14a, the second culture tank 14b, and the third culture tank 14c. That is, the supply step S2 of supplying the mixed gas to the first culture tank 14a is performed. In the first embodiment, the digestate produced as a by-product in the methane fermentation tank 12 is supplied to the first culture tank 14a, the second culture tank 14b, and the third culture tank 14c via a route different from the first supply line 20 and the second supply line 28. As described above, the digestate contains inorganic substances such as phosphorus, nitrogen, and potassium.

Specifically, the mixed gas produced in the methane fermentation tank 12 flows through the first supply line 20. As described above, the first supply line 20 is connected to the main pipe 22 in the first water tank 13a. A plurality of sub-pipes 24 branch from the main pipe 22. In the first culture tank 14a, guide pipes 25 are positioned above the sub-pipes 24. Therefore, in the supply step S2, the mixed gas flows into the water 26 (culture solution) from the first supply line 20 through the main pipe 22 and the sub-pipes 24 and then flows into the guide pipes 25.

As noted above, the culture solution is water 26. The solubility of carbon dioxide in water 26 is significantly greater than that of methane in water 26. Thus, while the mixed gas flows out from the sub-pipes 24 into the water 26 and goes up through the water 26, the carbon dioxide in the mixed gas is preferentially dissolved in the water 26. That is, part of carbon dioxide is removed from the mixed gas. Therefore, the concentration of methane in the mixed gas increases. The mixed gas with increased methane concentration is stored in a space between the water surface and the ceiling wall of the first culture tank 14a.

As described above, carbon dioxide has been dissolved in the water 26 in the first culture tank 14a. In the first culture tank 14a, this carbon dioxide is fixed to algae. Algae rely on the fixed carbon dioxide for photosynthesis. Thus, the culture step S3 is started in the first culture tank 14a. In this culturing step S3, carbon dioxide in the culture solution is consumed. Further, the algae take in, as nutrients, inorganic substances such as phosphorus, nitrogen, and potassium contained in the digestate.

The mixed gas stored in the space is supplied to the second culture tank 14b via the second supply line 28. The second supply line 28 is connected to the main pipe 22 in the second water tank 13b. The plurality of sub-pipes 24 branch from the main pipe 22, and the guide pipes 25 are positioned above the sub-pipes 24 in the second culture tank 14b. Therefore, the mixed gas flows into the water 26 from the second supply line 28 through the main pipe 22 and the sub-pipe 24 and then flows into the guide pipes 25. Thus, the re-supply step S4 is started in the second culture tank 14b.

The mixed gas flowing out from the guide pipes 25 ascends in the water 26. In this process, carbon dioxide is preferentially dissolved in the water 26 as in the first culture tank 14a. That is, carbon dioxide is further removed from the mixed gas. Accordingly, the methane concentration in the mixed gas further increases. The mixed gas having a further increased methane concentration is stored in a space between the water surface and the ceiling wall of the second culture tank 14b.

As described above, carbon dioxide has been dissolved in the water 26 in the second culture tank 14b. In the second culture tank 14b, this carbon dioxide is fixed to algae. Algae rely on the fixed carbon dioxide for photosynthesis. As described above, the re-supply step S4 in the first embodiment refers to re-supplying the mixed gas, which has been supplied to one culture tank and reduced in carbon dioxide, to another culture tank to culture algae. In this re-supply step S4, carbon dioxide in the culture solution is consumed. Further, the algae take in, as nutrients, inorganic substances such as phosphorus, nitrogen and potassium contained in the digestate.

The mixed gas stored in the space is supplied to the third culture tank 14c via the second supply line 28 of the second culture tank 14b. The second supply line 28 is connected to the main pipe 22 in the third water tank 13c. A plurality of sub-pipes 24 branch from the main pipe 22, and the guide pipes 25 are positioned above the sub-pipes 24 in the third culture tank 14c. Therefore, the mixed gas flows into the water 26 from the second supply line 28 via the main pipe 22 and the sub-pipes 24 and then flows into the guide pipes 25. Thus, the re-supply step S4 is started in the third culture tank 14c.

The mixed gas flowing out from the guide pipes 25 into the culture solution ascends in the water 26. In this process, carbon dioxide is preferentially dissolved in the water 26 as in the first culture tank 14a and the second culture tank 14b. That is, carbon dioxide is further removed from the mixed gas. Accordingly, the methane concentration in the mixed gas further increases, and methane of high purity can be obtained. In this way, methane is purified. The high-purity methane is stored in a space between the water surface and the ceiling wall of the third culture tank 14c.

The high-purity methane flows through the collection line 30 provided at the ceiling wall of the third culture tank 14c and flows into the collection tank 16. That is, the high-purity methane is recovered by the collection tank 16. The obtained methane is used, for example, as a fuel.

As described above, carbon dioxide has been dissolved in the water 26 in the third culture tank 14c. Also in the third culture tank 14c, this carbon dioxide is fixed to algae. Algae rely on the fixed carbon dioxide for photosynthesis. As a result, culture is performed in the third culture tank 14c. By this culture (re-supply step S4), carbon dioxide in the culture solution is consumed. Further, the algae take in, as nutrients, inorganic substances such as phosphorus, nitrogen and potassium contained in the digestate.

As described above, the carbon dioxide dissolved in the culture solutions of the first to third culture tanks 14a to 14c is used for photosynthesis of algae. Therefore, carbon dioxide is prevented from being discharged from the first to third culture tanks 14a to 14c. Thus, in the first embodiment, the amount of carbon dioxide discharged from the methane purification apparatus 10 reduces. Accordingly, the first embodiment contributes to the climate change mitigation or impact reduction.

As understood from the above, in the first embodiment, it is not necessary to provide the absorption tower described in JP 2010-088368 A. Therefore, the size of the methane purification apparatus 10 can be reduced. Further, capital investment reduces.

The cultured algae are collected together with the culture solution from the first to third culture tanks 14a to 14c. The collected algae and culture solution are introduced into the flash distiller 18 shown in FIG. 2. Specifically, the pump 34 is driven. With this driving, the respective culture solutions and algae in the first to third culture tanks 14a to 14c are sent to the flash distiller 18 via the solution feed line 32. The collection step S5 is carried out in the flash distiller 18.

The interior of the flash distiller 18 is depressurized in advance. In this environment, the culture solution vaporizes. From the vaporized culture solution (water vapor), methane slightly dissolved in the culture solution is separated. Methane and water vapor enter the exhaust line 36 from the flash distiller 18. The water vapor is condensed at the condenser 38 provided on the exhaust line 36. The condensed water is sent to the flash distiller 18 via the return line 40. That is, in the condenser 38, water vapor is removed from the mixed gas of methane and water vapor. Thus, methane of high purity can be obtained.

The exhaust line 36 is connected, for example, to the collection tank 16. Therefore, the high-purity methane that has passed through the exhaust line 36 is collected in the collection tank 16. Thus, a large amount of high-purity methane can be collected.

In the flash distiller 18, the culture solution is separated into a sediment 50 in which algae have settled and a supernatant 52. Sediment 50 may be withdrawn from the bottom of the flash distiller 18. The sediment 50 extracted from the flash distiller 18 can be used as organic residue for generating methane in the methane fermentation tank 12. As a result, the cultured algae can be effectively utilized. The supernatant 52 may be collected from the flash distiller 18, adjusted in composition to be suitable for culturing, and then reused for culturing algae.

The number of culture tanks in the first embodiment is not limited to three. The number of culture tanks may be two or four or more. In any case, one culture tank is connected to another culture tank by the second supply line 28. In this configuration, the mixed gas from the methane fermentation tank 12 is first supplied to one culture tank. Thereafter, the mixed gas that has passed through the culture tank is supplied to another culture tank.

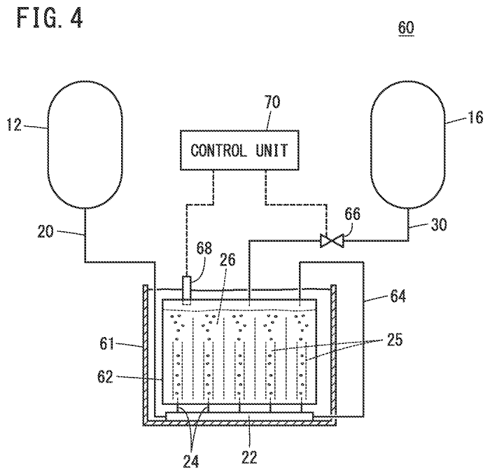

Next, a second embodiment will be described with reference to FIG. 4. The same components as those shown in FIGS. 1 and 2 are denoted by the same reference numerals, and detailed description thereof is omitted.

FIG. 4 is a schematic system diagram of an essential part of a methane purification apparatus 60 according to the second embodiment. The number of culture tanks in the methane purification apparatus 10 according to the first embodiment is plural. On the other hand, the number of culture tanks in the methane purification apparatus 60 according to the second embodiment is one. In order to easily distinguish between the first embodiment and the second embodiment, in the second embodiment, the water tank is given reference numeral “61” and the culture tank is given reference numeral “62”.

The culture tank 62 is immersed in water in the water tank 61. The culture tank 62 is provided with a circulation supply line 64 as a second supply line, and a collection line 30. The collection line 30 is connected to the collection tank 16 as in the first embodiment. The collection line 30 is provided with an on-off valve 66. In addition, a methane detection sensor 68 is provided at a ceiling wall of the culture tank 62. The on-off valve 66 and the methane detection sensor 68 are electrically connected to a control unit 70. It is to be noted that the methane purification apparatus 60 includes the flash distiller 18 similar to the methane purification apparatus 10 but the flash distiller 18 is omitted in FIG. 4.

In the purification method according to the second embodiment, the mixed gas starting from the methane fermentation tank 12 flows into the water 26 via the first supply line 20, the main pipe 22, and the sub-pipes 24. The mixed gas having slightly ascended in the water 26 flows into the guide pipes 25 and ascends in the guide pipes 25. In this process, carbon dioxide contained in the mixed gas is preferentially dissolved in the water 26 (culture solution). That is, carbon dioxide is removed from the mixed gas. Therefore, the concentration of methane in the mixed gas increases. The mixed gas having an increased methane concentration is stored in a space between the water surface and a ceiling wall of the culture tank 62. Meanwhile, the on-off valve 66 provided on the collection line 30 is in a closed state. Therefore, at this stage, the mixed gas does not flow to the collection tank 16 through the collection line 30.

The mixed gas stored in the space is re-supplied to the culture tank 62 via the circulation supply line 64. That is, the mixed gas flows out from the circulation supply line 64 into the water 26 via the main pipe 22, the sub-pipes 24 and the guide pipes 25. After this re-supply is started, the outflow of the mixed gas from the main pipe 22 may be stopped.

As described above, while the mixed gas is ascending through the water 26, carbon dioxide contained in the mixed gas is preferentially dissolved in the water 26. That is, carbon dioxide is further removed from the mixed gas. Accordingly, the methane concentration in the mixed gas further increases. The mixed gas having a further increased methane concentration is stored in the space between the water surface and the ceiling wall of the culture tank 62.

Thereafter, by repeating the circulation supply described above, carbon dioxide is successively removed from the mixed gas. Accordingly, high-purity methane is stored in the space. The control unit 70 acquires the methane concentration based on a detection result at the methane detection sensor 68.

When the methane concentration reaches a predetermined threshold value, the control unit 70 issues a command signal for opening the on-off valve 66. As a result, the on-off valve 66 opens. Thus, high-purity methane is collected in the collection tank 16. As described above, even when the number of culture tanks is one, it is possible to obtain high-purity methane.

Also in the culture tank 62, algae reply on the carbon dioxide dissolved in the water 26 for photosynthesis. Further, the algae take in, as nutrients, inorganic substances (phosphorus, nitrogen, potassium or the like) produced in the methane fermentation tank 12 and supplied in advance to the culture tank 62.

As in the first embodiment, the cultured algae are supplied to the flash distiller 18 (see FIGS. 1 and 2) together with the culture solution. High-purity methane can be collected also by this flash distillation.

According to the second embodiment, since the number of culture tanks is smaller than that of the first embodiment, the methane purification apparatus 60 can be made smaller than the methane purification apparatus 10.

As described above, the present embodiment discloses the methane purification method for purifying methane from the mixed gas containing carbon dioxide and methane by removing carbon dioxide from the mixed gas, including the methane fermentation step (S1) of performing methane fermentation for organic residue, a supply step (S2) of supplying the mixed gas containing methane and carbon dioxide discharged in the methane fermentation step to the culture tank containing water (26) as a culture solution for culturing algae, the culture step (S3) of culturing the algae by fixing the carbon dioxide contained in the mixed gas to the algae, and the re-supply step (S4) of re-supplying to the culture tank or another culture tank the mixed gas that has passed through the culture solution to reduce the carbon dioxide contained in the mixed gas, wherein the carbon dioxide contained in the mixed gas is consumed by performing the re-supply step at least once.

Thus, in the present embodiment, the mixed gas containing methane and carbon dioxide is supplied to the algae. As a result, carbon dioxide is fixed to the algae and thus carbon dioxide reduces in the mixed gas. This mixed gas is re-supplied to the algae. By this resupply, carbon dioxide in the mixed gas further reduces. Therefore, the purity of methane increases. That is, methane of high purity can be obtained. Also, carbon dioxide is fixed to the algae and consumed through the photosynthesis of the algae. As a result, the present invention reduces carbon dioxide and thus contributes to climate change mitigation or impact reduction.

In addition, an absorption tower for absorbing the mixed gas into water is unnecessary. Therefore, it is possible to reduce the size of the methane purification apparatus. Further, it is easy to reduce capital investment.

The present embodiment discloses the methane purification method wherein a plurality of culture tanks (14a to 14c) includes the culture tank, and the mixed gas that has passed through the culture solution in one of the culture tanks is supplied to the culture solution in another one of the culture tanks, and the re-supply step is performed in the another culture tank.

In this case, the mixed gas sequentially passes through the plurality of culture tanks, whereby the carbon dioxide contained in the mixed gas can be successively reduced.

The present embodiment discloses the methane purification method wherein the culture tank is a single culture tank (62), and the mixed gas that has passed through the culture solution in the single culture tank is supplied to the culture solution in the single culture tank, and the re-supply step is performed with the single culture tank.

In this case, by supplying the mixed gas to a single culture tank in a circulated manner, it is possible to successively reduce carbon dioxide contained in the mixed gas.

The present embodiment discloses the methane purification method for purifying methane wherein an inorganic substance contained in residue produced in the methane fermentation step is supplied to the culture tank or the another culture tank.

The digestate produced as a by-product of methane fermentation contains inorganic substances such as phosphorus, nitrogen, and potassium. These inorganic substances become nutrients for algae. Therefore, by supplying the inorganic substances to the culture tank, it is possible to promote the culture of algae.

The present embodiment discloses the methane purification method wherein the methane fermentation step is performed using, as the organic residue, the algae cultured in the culturing step or the re-supplying step.

Thus, the cultured algae can be effectively utilized. Accordingly, it is possible to reduce the amount of cultured algae to be discarded.

The present embodiment discloses the methane purification method further including the collection step (S5) of collecting the culture solution containing the algae after the culture step or the re-supply step, wherein in the collection step, methane is separated from the culture solution through flash distillation.

The culture solution contains a small amount of dissolved methane. Flash distillation makes it possible to separate gas from liquid. Accordingly, by carrying out the collection step described above, it is possible to separate a small amount of methane dissolved in the culture solution from the algae and the culture solution. Thus, the amount of methane that is recovered from the mixed gas produced by methane fermentation increases.

The present embodiment discloses the methane purification apparatus (10) that purifies methane from the mixed gas containing carbon dioxide and methane by removing the carbon dioxide from the mixed gas, the apparatus including the methane fermentation tank (12) that performs methane fermentation for organic residue, the culture tank that holds water as a culture solution for culturing algae, the first supply line (20) that supplies the culture tank with the mixed gas containing methane and carbon dioxide discharged from the methane fermentation tank, and the second supply line (28) that supplies another culture tank different from the culture tank with the mixed gas that has passed through the culture solution.

With this configuration, high-purity methane can be obtained. Further, carbon dioxide reduces because carbon dioxide is fixed to algae and is consumed through photosynthesis of the algae. Therefore, the present invention can contribute to climate change mitigation or impact reduction.

In addition, since an absorption tower for absorbing the mixed gas into water is unnecessary, the size of the methane purification apparatus can be reduced. Further, it is easy to reduce capital investment.

The present embodiment discloses the methane purification apparatus, further comprising a plurality of culture tanks (14a to 14c) including the culture tank, wherein the second supply line supplies the mixed gas from one of the culture tanks to another one of the culture tanks.

In this case, the mixed gas sequentially passes through the plurality of culture tanks, whereby the carbon dioxide contained in the mixed gas can be successively reduced.

The present embodiment discloses the methane purification apparatus wherein the culture tank is the single culture tank (62), and the second supply line supplies the mixed gas in a circulated manner from the single culture tank to the single culture tank.

In this case, by supplying the mixed gas to a single culture tank in a circulated manner, it is possible to successively reduce carbon dioxide contained in the mixed gas.

The present embodiment discloses the methane purification apparatus including the flash distiller (18) that performs flash distillation for the culture solution containing the algae.

A small amount of methane dissolved in the culture solution can be separated from algae and the culture solution with a flash distiller. As a result, the amount of methane recovered from the mixed gas produced by methane fermentation is increased.

The present invention is not limited to the above-described embodiments, and various configurations can be adopted therein without departing from the essence and gist of the present invention.

For example, food waste or the like may be used as the organic residue.

What is claimed is:

1. A methane purification method of purifying methane from a mixed gas containing carbon dioxide and methane by removing the carbon dioxide from the mixed gas, the method comprising:
performing methane fermentation for organic residue;
supplying a mixed gas containing methane and carbon dioxide discharged in performing the methane fermentation to a culture tank containing water as a culture solution for culturing algae;
culturing the algae by fixing the carbon dioxide contained in the mixed gas to the algae; and
re-supplying to the culture tank or another culture tank the mixed gas that has passed through the culture solution to reduce the carbon dioxide contained in the mixed gas,
wherein
the carbon dioxide contained in the mixed gas is consumed by performing the re-supplying at least once.

2. The methane purification method according to claim 1,
wherein
a plurality of culture tanks includes the culture tank, and
the mixed gas that has passed through the culture solution in one of the culture tanks is supplied to the culture solution in another one of the culture tanks, and the re-supplying is performed in the another one of the culture tanks.

3. The methane purification method according to claim 1,
wherein
the culture tank is a single culture tank, and
the mixed gas that has passed through the culture solution in the single culture tank is re-supplied to the culture solution in the single culture tank, and the re-supplying is performed with the single culture tank.

4. The methane purification method according to claim 1, wherein
an inorganic substance contained in residue produced in performing the methane fermentation is supplied to the culture tank or the another one of the culture tanks.

5. The methane purification method according to claim 1, wherein
the methane fermentation is performed using, as the organic residue, the algae cultured in the culturing or the re-supplying.

6. The methane purification method according to claim 1, further comprising
collecting the culture solution containing the algae after the culturing or the re-supplying,
wherein in the collecting, the methane is separated from the culture solution through flash distillation.

7. A methane purification apparatus that purifies methane from a mixed gas containing carbon dioxide and methane by removing the carbon dioxide from the mixed gas, the apparatus comprising:
a methane fermentation tank that performs methane fermentation for organic residue;
a culture tank that holds water as a culture solution for culturing algae;
a first supply line that supplies the culture tank with a mixed gas containing methane and carbon dioxide discharged from the methane fermentation tank; and
a second supply line that supplies another culture tank different from the culture tank with the mixed gas that has passed through the culture solution.

8. The methane purification apparatus according to claim 7, further comprising a plurality of culture tanks including the culture tank,
wherein the second supply line supplies the mixed gas from one of the culture tanks to another one of the culture tanks.

9. The methane purification apparatus according to claim 7, wherein the culture tank is a single culture tank, and
the second supply line supplies the mixed gas in a circulated manner from the single culture tank to the single culture tank.

10. The methane purification apparatus according to claim 7, further comprising a flash distiller that performs flash distillation for the culture solution containing the algae.
